(19) United States
(12) Patent Application Publication
(10) Pub. No.: US 2003/0064086 A1
(43) Pub. Date: Apr. 3, 2003

(54) Cosmetic compositions comprising nanoparticles and processes for using the same

(76) Inventors: Danuvio Carrion, Fords, NJ (US); Debra Coleman-Nally, Neshanic, NJ (US); Gary Graves, Morganville, NJ (US)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/231,139

(22) Filed: Aug. 30, 2002

Related U.S. Application Data
(60) Provisional application No. 60/315,957, filed on Aug. 31, 2001.

(51) Int. Cl. A61K 7/021; A61K 7/00

(52) U.S. Cl. 424/401; 424/63

(57) ABSTRACT

A cosmetic composition comprising nanoparticles in a cosmetically acceptable medium. A process for making up, caring for and/or treating at least one keratinous substance comprising applying a cosmetic composition comprising nanoparticles in a cosmetically acceptable medium.

This is application claims the benefit of U.S. provisional application No. 60/315,957, filed Aug. 31, 2001, which is incorporated herein by reference.

The present invention relates to a cosmetic composition for at least one keratinous substance comprising nanoparticles in a cosmetically acceptable medium, wherein the nanoparticles are present in an amount effective to impart at least one long wear property to the cosmetic composition, wherein the nanoparticles do not decrease the gloss of the composition, and methods for using these compositions.

Make-up compositions may be colored with the aid of dyestuffs such as soluble dyes and pigments such as metal oxides (e.g., iron oxide) and/or with the aid of nacres such as micas coated with metal oxides (e.g., titanium oxide). These compositions may further comprise a film former which may increase the composition's resistance to wear. However, when a film of such a make-up composition is subjected to stresses such as impacts, pressure or rubbing, the dyestuffs may not be resistant to these stresses and thus may wear away. A loss of color of the make-up may then be observed, thus obliging the user to reapply the make-up composition regularly, optionally after the damaged film has been removed.

Make-up compositions may comprise discrete elements which may improve the composition's durability. Non-limiting examples of such discrete elements include fibers, diamond, and silica. However, the incorporation of such elements may result in a film which has a rough texture with little gloss.

Thus, in one embodiment, the present invention is directed to a cosmetic composition for at least one keratinous substance comprising nanoparticles in a cosmetically acceptable medium, wherein the nanoparticles are present in an amount effective to impart at least one long wear property to the cosmetic wherein the nanoparticles do not decrease the gloss of the composition. As used herein, the term “nanoparticles” refers to at least one type of nanoparticles, and thus may include nanoparticles formed from a single material, nanoparticles formed from a combination of materials, as well as a mixture of nanoparticles, each formed from at least one different material.

In another embodiment, the present invention provides a process for making up at least one keratinous substance comprising applying to the at least one keratinous substance a cosmetic composition comprising nanoparticles in a cosmetically acceptable medium, wherein the nanoparticles are present in an amount effective to impart at least one long wear property to the cosmetic composition, wherein the nanoparticles do not decrease the gloss of the composition.

In another embodiment, the present invention is drawn to a method for caring for or treating at least one keratinous substance comprising applying to the at least one keratinous substance a cosmetic composition comprising nanoparticles in a cosmetically acceptable medium, wherein the nanoparticles are present in an amount effective to impart at least one long wear property to the cosmetic composition, wherein the nanoparticles do not decrease the gloss of the composition.

In yet another embodiment, the present invention is drawn to a process for imparting at least one long wear property to a cosmetic composition comprising including nanoparticles in a cosmetic composition and applying the cosmetic composition to the at least one keratinous substance.

In yet another embodiment, the present invention is drawn to a process for maintaining the gloss of a composition for at least one keratinous substance comprising including nanoparticles in a cosmetic composition and applying the cosmetic composition to the at least one keratinous substance.

Certain terms used herein are defined below:

“At least one” as used herein means one or more and thus includes individual components as well as mixtures/combinations.

“Alkyl” as used herein, refers to substituted linear alkyl groups, unsubstituted linear alkyl groups, substituted branched alkyl groups, unsubstituted branched alkyl groups, substituted cyclic alkyl groups and unsubstituted cyclic alkyl groups, wherein the alkyl groups comprise at least one carbon and may optionally further comprise at least one heteroatom intercalated in the alkyl chain.

“Substituted,” as used herein, means comprising at least one substituent. Non-limiting examples of substituents include atoms, such as halogen atoms, oxygen atoms and nitrogen atoms, as well as functional groups, such as hydroxyl groups, ether groups, oxyalkylene groups, polyoxyalkylene groups, carboxylic acid groups, amine groups, amide groups, halogen containing groups, ester groups, siloxane groups, and polysiloxane groups.

A “concentrated layer of nanoparticles,” as used herein, refers to a region or layer parallel to the surface of a substrate to which the inventive composition is applied and which comprises a higher concentration of the nanoparticles than at least one other region or layer of the film which is parallel to the substrate surface. Accordingly, the phrase “nanoparticles form a concentrated layer of nanoparticles in the cosmetic composition on the at least one keratinous substance” does not exclude nanoparticles from also being present in the cosmetic composition outside the concentrated layer.

A “coating,” as used herein, refers to a layer of composition deposited on a substrate which covers at least a portion of the substrate.

A “film,” as used herein, refers to a continuous coating, i.e., a coating without holes visible to the naked eye, which covers at least a portion of the substrate to which the composition was applied. Further, a film, as used herein, may have any thickness and is not restricted to a thin coating.

“Film-forming polymer” as used herein means a polymer which, by itself or in the presence of a film-forming auxiliary, is capable, after dissolution in at least one solvent, of forming a film on the substrate to which it is applied once the at least one solvent evaporates.

“Gloss” as used herein, refers to surface shininess. The “nanoparticles do not decrease the gloss of a composition” which comprises nanoparticles according to the present invention when there is no measurable decrease in the gloss of the composition on at least one substrate upon inclusion of nanoparticles in the composition. In one embodiment, the gloss of a composition comprising nanoparticles on at least one substrate following application of the composition to the substrate is at least the same as the gloss of the same composition but which does not comprise nanoparticles on the at least one substrate following application to the substrate. In one embodiment, the gloss of a composition comprising nanoparticles on at least one substrate following application of the composition to the substrate is greater than the gloss of the same composition but which does not comprise nanoparticles on the at least one substrate following application to the substrate. The gloss of a composition according to the present invention is measured and evaluated using a gloss meter. See Example 1. Gloss meters are commonly used in the nail polish art, and measure the amount of light reflected from the surface, coating or film of interest. The gloss may be quantified, for example, as a % reflectance.

“Hardness,” as used herein, refers to the resistance of a film or a coating of a cosmetic composition to scratching and abrasion. Hardness, as used herein, is tested by the Persoz Pendulum Hardness Test (ASTM D4366-95)

“Long wear properties” as used herein, refers to at least one property chosen from gloss, color, concentration, consistency, and texture on the at least one keratinous substance that remains the same after an extended period of time as at the time of application, as viewed by the naked eye. Thus, “long wear properties” include resistance to at least one of impacts, rubbing, scuffing, abrasion, and chipping. One of ordinary skill in the art will recognize that the length of the extended period of time will depend on the application of the inventive composition and may range up to, for example, 1 hour, such as 2 hours, and further such as 8 hours. For example, the length of the extended period of time for a face powder, lip composition, eyeshadow, foundation, eyeliner or mascara is up to 8 hours. For example, the length of the extended period of time for a nail varnish is up to 3 days. In one embodiment, the length of the extended period of time for a nail varnish is up to 5 days, and further such as up to 8 days.

Long wear properties may be evaluated by any method known in the art for evaluating such properties, and which will, of course, depend on the application of the inventive composition. For example, long wear properties of a composition intended for application to skin (including lips) is evaluated by a test involving the application of the composition to human skin (and/or lips) and evaluating at least one of the consistency, texture and color of the composition after an extended period of time. For example, the consistency, texture and color of a lip composition may be evaluated immediately following application and these characteristics may then be re-evaluated and compared after an individual has worn the lip composition for a certain amount of time. Further long wear properties of a nail varnish is evaluated by a test involving the resistance to at least one of impacts, rubbing, scuffing, abrasion, and/or chipping of a composition and evaluating the gloss, texture and concentration remaining on the nail after testing. Finally, the characteristics may be evaluated with respect to other compositions, such as commercially available compositions. Long wear may, for example, be tested by visual observation in live models. For example, the fingernails of a live model may be coated with a control and a test nail polish and the long wear properties compared.

The long wear properties of a nail varnish is measured by an objective wear test. For example, the wear resistance of the films is measured according to AFNOR standard NF T 30-015. Each composition is applied in the form of a coat 600 μm thick (before drying) onto a disc and then left to dry for 1 hour at 30° C. The film of varnish deposited on the disc is then placed for 1 hour in contact with abrasive discs (Taber abrasimeter), the disc making a complete rotation in one second. After 1 hour, the disc is weighed and the loss of mass LM of product, expressed as a percentage of the weight lost relative to the initial weight, is calculated.

“Keratinous substance” as defined herein may be human keratinous substances, and may be chosen from, for example, nails, facial skin (including the lips), body skin, and keratinous fibers such as eyelashes, eyebrows, and hair.

“Polymers” as defined herein comprise copolymers (including terpolymers) and homopolymers, including but not limited to, for example, block polymers, cross linked polymers, and graft polymers.

“Polycondensation” as used herein refers to a polymerization reaction which comprises at least one condensation reaction, i.e., elimination of at least one water molecule, when at least two monomers, which may be identical or different, react with each other.

“Radical-mediated film-forming polymer” as used herein means a polymer obtained by a polymerization reaction which is initiated and/or propagated by at least one free radical. For example, this polymerization may occur by addition, in which the at least one free radical initiates polymerization by reaction with at least one monomer chosen from monomers comprising at least one unsaturated bond, such as an ethylene group, and monomers which are capable of homopolymerizing. By this mechanism, the polymer chain may become self-propagating.

“Rheological agent” as used herein refers to a molecule or a composition which can change, i.e., increase or decrease, at least one property chosen from deformation and flow, in terms of stress, strain and/or time, of a composition to which the Theological agent is added.

“Room temperature” as used herein refers to 25° C.

“Substrate,” as used herein, includes, for example, a keratinous material, a false nail, a false eyelash, a pastille, a patch for adhering to the skin or the lips, a wig, and any other surface to which a composition may be applied.

It is to be understood that both the foregoing general description and the following detailed description are exemplary and explanatory only and are not restrictive of the invention, as claimed. Reference will now be made in detail to exemplary embodiments of the present invention.

When the inventive cosmetic composition comprising nanoparticles is applied to at least one keratinous substance such as the nails, the nanoparticles may be distributed within the film deposited on the at least one keratinous substance. While not to be limited by theory, it is believed that the nanoparticles migrate after the cosmetic composition is applied to the at least one keratinous substance, for example towards the surface of the film, i.e., the air-composition interface. The film of the inventive cosmetic composition may, therefore, comprise a region, or layer, parallel to the substrate surface which comprises a higher concentration of the nanoparticles, i.e., a larger number of the nanoparticles, than at least one other region of the film parallel to the substrate surface, such as the region comprising the composition-keratinous substance interface, and further such as all other regions of the film. For example, a film of the inventive cosmetic composition may comprise a region parallel to the substrate surface at and/or near the air-composition interface, for example, wherein this region comprises a higher concentration of nanoparticles than the concentration of nanoparticles in the bulk region of the film, i.e., the region which extends beneath the surface region, such as, for example, the region at and/or near the composition-keratinous substance interface, and further such as, for example, the region between the air-composition interface and the composition-keratinous substance interface.

Thus, the inventive compositions may afford a film which has a very glossy appearance and which may have good long wear properties such as long wear properties chosen from resistance to impacts, rubbing, scuffing, abrasion, and/or chipping.

In one embodiment, the nanoparticles are present in the inventive composition in an amount effective to impart at least one long wear property to the inventive composition, wherein the nanoparticles do not decrease the gloss of the composition. In another embodiment, the inventive composition forms a glossy film on at least one keratinous substance, which film adheres well to the at least one keratinous substance and has at least one long wear property.

Nanoparticles (sometimes referred to as “nanocrystalline materials,” nanomaterials,” or “ceramic”) are materials that have an average particle size, or grain size, generally of less than 1 micron (1μ), such as less than 800 nanometers (800 nm, such as less than 500 nanometers (500 nm), and further such as less than 100 nanometers (100 nm). In one embodiment, the nanoparticles have an average particle size generally ranging from 1 nm to less than 1000 nm, such as from 10 nm to 500 nm, and further such as from 30 nm to 100 nm. The nanoparticles of the present invention are discrete particles and may be used in combination with non-nanoparticles, such as, for example, a mixture of nanoparticles with particles of a larger size.

The concentration of the nanoparticles in the inventive composition as well as in the above-defined regions and the size of the nanoparticles can be measured by methods known for such which are well known in the art. For example, x-ray diffraction (XRD), scanning electron microscopy (SEM), transmission electron microscopy (TEM), and/or BET surface area analysis may be used. In one embodiment, the nanoparticles of the present invention are chosen from spherical nanoparticles, spheroidal nanoparticles and tubular nanoparticles. In another embodiment, the nanoparticles are not chosen from nanoparticles in platelet form. In another embodiment, the nanoparticles are chosen from hollow nanoparticles and solid nanoparticles. In yet another embodiment, the nanoparticles are chosen from solid nanoparticles, i.e., not hollow nanoparticles.

In another embodiment, the nanoparticles may be chosen from at least one metallic powder such as metallic powders formed from at least one metal; at least one inorganic powder such as inorganic powders formed from at least one inorganic oxide; at least one nanoparticle formed from at least one composite polymer; at least one nanoparticle formed from at least one clay; and mixtures of any of the foregoing. As used herein, the phrase “formed from”, is open ended and does not limit the components of the nanoparticles. In one embodiment, the nanoparticles of the present invention are discrete nanoparticles. In one embodiment, the discrete nanoparticles remain separate nanoparticles following film formation. In one embodiment, the nanoparticles of the present invention do not significantly affect the viscosity of the composition. In one embodiment, the nanoparticles of the present invention are not thixotropes.

In one embodiment, the nanoparticles of the present invention do not include nanoparticles formed only from at least one polymer, i.e., polymeric particles or at least one polymer dispersed in the form of particles. In one embodiment, the nanoparticles of the present invention are not chosen from nanoparticles of polyester-polyurethane and nanoparticles of polyether-polyurethane. In one embodiment, the nanoparticles of the present invention are not chosen from nanoparticles of acrylic polymers. In one embodiment, the nanoparticles of the present invention do not include uncoated nanoparticles comprised of at least 10% alumina having a particle size ranging from 5 nm to 25 nm. In one embodiment, the nanoparticles of the present invention do not include nanoparticles formed from colloidal silicic acid (such as hollow spherical silica nanoparticles and fumed silica nanoparticles). In one embodiment, the nanoparticles of the present invention do not include titanium dioxide nanoparticles. In one embodiment, the nanoparticles of the present invention do not include zinc oxide nanoparticles. A mixture of nanoparticles of the present invention with nanoparticles chosen from at least one of nanoparticles formed only from at least one polymer, nanoparticles formed from colloidal silicic acid, titanium dioxide nanoparticles, and zinc oxide nanoparticles are within the practice of the invention. In one embodiment, the nanoparticles are not chosen from hollow inorganic microspheres with an average size of 500 nm and greater and having a shell of fused and at least partially crystallized material.

Non-limiting examples of metallic powders formed from at least one metal include metallic powders formed from silver, metallic powders formed from gold, metallic powders formed from platinum, metallic powders formed from palladium, metallic powders formed from nickel, and mixtures of any of the foregoing. Non-limiting examples of inorganic powders formed from at least one inorganic oxide include oxides of magnesium, oxides of calcium, oxides of strontium, oxides of yttrium, oxides of chromium, oxides of molybdenum, oxides of vanadium, oxides of titanium, oxides of holmium, oxides of cerium, oxides of neodymium, oxides of iron, oxides of copper, oxides of aluminum, oxides of indium, oxides of silicon, oxides of tin, oxides of antimony, and mixtures of any of the foregoing.

In one embodiment, the nanoparticles may be chosen from coated nanoparticles. In one embodiment, the nanoparticles may be chosen from nanoparticles coated with at least one material chosen from materials that will change the polarity of the nanoparticles and materials that will change the hydrophobicity of the nanoparticles. Such coatings may control the dispersion, agglomeration, re-agglomeration and/or migration of the nanoparticles. A change in the hydrophobicity of the nanoparticles refers to a measurable increase or a measurable decrease in the nanoparticles' affinity for water, compatibility with water, and/or capability to be dissolved in water as compared to that of uncoated nanoparticles formed from the same material(s). Methods for measuring the hydrophobicity of molecules and particles are well known in the art and may be used to measure any change in hydrophobicity of the nanoparticles. Similarly, a change in the polarity of the nanoparticles refers to a measurable increase or a measurable decrease in the dipole moment of the nanoparticles as compared to that of uncoated nanoparticles formed from the same material(s). As with the hydrophobicity of the nanoparticles, methods for measuring the polarity of molecules and particles are well known in the art and may be used to measure any change in polarity of the nanoparticles.

In another embodiment, the coated nanoparticles may be chosen from nanoparticles coated with at least one material that will lower the surface free energy of the nanoparticles, i.e., with at least one material that will increase the difference between the surface tension of the nanoparticles and the surface tension of the at least one cosmetically acceptable medium of the inventive composition. Methods for measuring surface tension changes are well known in the art and may be used to measure any change in the surface tension of the nanoparticles and the cosmetically acceptable medium. Non-limiting examples of the at least one material that will lower the surface free energy of the nanoparticles include phosphates such as triphenyl phosphate; fluorinated compounds such as perfluoroethers and derivatives thereof; silicone compounds such as polysilicone-8 which is commercially available under the tradename VS 80 from 3M company; and hydrocarbons such as polyethylene.

Non-limiting examples of coated and uncoated nanoparticles suitable for use in the present invention are those that are commercially available from manufacturers such as Nanocor (Arlington Heights, Ill.), Montell North America (Wilmington, Del.), Nanopowder Industries (Kfar-Saba, Israel), H. C. Stark GmbH & Co., K. G. (Goslar, Germany), Vista Chemical Company (Houston, Tex.), Nanotechnologies (Austin, Tex.), Nanomat (North Huntingdon, Pa.), Nanophase Technologies Corporation, (Burr Ridge, Ill.), Argonide Corporation (Sanford, Fla.), Reade Advanced Materials (Providence, R.I.), Nanomaterials Research Corporation (Longmont, Colo.).

In one embodiment, nanoparticles are chosen from nanoparticles produced by one of the at least five methods that are known for producing nanoparticles. These include: sol-gel synthesis; inert gas condensation; mechanical alloying and high-energy ball milling; plasma synthesis; and electrodeposition. These methods produce nanoparticles in commercially-viable quantities in varying degrees. Methods of manufacturing are described in several publications such as Chemical Enqineering, April 1999, Vol. 106, Issue 4, p. 106, which page is incorporated herein by reference; and Ceramic Industry 1998 Materials Handbook, which methods disclosed therein are incorporated herein by reference. Systems and methods for making nanoparticles are described, for example, in U.S. Pat. Nos. 5,984,997; 5,075,090; 5,358,695; 5,958,361; 5,407,458; 5,403,375; 5,514,349; 5,874,684; 5,460,701; and 5,851,507. Similarly, coated nanoparticles and methods of making them are described in U.S. Pat. Nos. 6,033,781, 5,993,967, 5,788,738, 6,228,904, and WO 00/09446.

In one embodiment of the invention, the nanoparticles are present in an amount generally ranging from 0.01% to 50% by weight, relative to the total weight of the composition, such as from 0.1% to 40%, such as from 1% to 30%, further such as from 1.5% to 20%, further such as from 2% to 10%, and even further such as from 3% to 8%.

The compositions according to the invention may further comprise at least one film-forming polymer. The at least one film-forming polymer may be dissolved or dispersed, for example, in the form of particles, in the cosmetically acceptable medium. Non-limiting examples of the at least one film-forming polymer include synthetic film-forming polymers including film-forming polymers formed via radical-mediated polymerization and film-forming polymers formed from polycondensation, and film-forming polymers of natural origin. In one embodiment, the compositions of the present invention do not include both an aqueous dispersion of at least one film forming polymer having a particle size ranging from 10 nm to 500 nm and at least one associative polyurethane.

Non-limiting examples of the at least one film-forming polymer formed via radical-mediated polymerization include vinyl polymers (including vinyl copolymers), such as, for example, acrylic polymers. For example, vinyl film-forming polymers may be formed from polymerization of at least one monomer comprising at least one ethylenically unsaturated group and at least one additional group chosen from acid groups, ester groups, and amide groups.

Non-limiting examples of at least one monomer comprising at least one ethylenically unsaturated group and at least one acid group include α,β-ethylenically unsaturated carboxylic acids such as acrylic acid, methacrylic acid, (meth)acrylic acid, crotonic acid, maleic acid and itaconic acid.

Non-limiting examples of at least one monomer comprising at least one ethylenically unsaturated group and at least one ester group include (meth)acrylates such as alkyl (meth)acrylates, aryl (meth)acrylates; and hydroxyalkyl (meth)acrylates. Non-limiting examples of alkyl (meth)acrylates include C₁-C₂₀ alkyl (meth)acrylates, such as C₁-C₈ alkyl (meth)acrylates, and further such as methyl methacrylate, ethyl methacrylate, butyl methacrylate, isobutyl methacrylate, 2-ethylhexyl methacrylate, and lauryl methacrylate. Non-limiting examples of aryl (meth)acrylates include C₆-C₁₀ aryl (meth)acrylates, such as benzyl acrylate and phenyl acrylate. Non-limiting examples of hydroxyalkyl (meth)acrylates include C₂-C₆ hydroxyalkyl (meth)acrylates, such as hydroxyethyl acrylate, 2-hydroxypropyl acrylate, hydroxyethyl methacrylate, and 2-hydroxypropyl methacrylate.

Non-limiting examples of at least one monomer comprising at least one ethylenically unsaturated group and at least one amide group include (meth)acrylamides, such as N-alkyl (meth)acrylamides, and further such as N-(C₂-C₁₂ alkyl) (meth)acrylamides (such as N-ethylacrylamide, N-tert-butylacrylamide, and N-tert-octylacrylamide).

Further for example, vinyl film-forming polymers may be formed from homopolymerization or copolymerization of at least one monomer chosen from vinyl esters and styrene monomers. Thus, for example, these monomers may be polymerized with at least one monomer comprising at least one ethylenically unsaturated group and at least one additional group chosen from acid groups, ester groups, and amide groups, such as those previously mentioned.

Non-limiting examples of vinyl esters include vinyl acetate, vinyl neodecanoate, vinyl pivalate, vinyl benzoate and vinyl tert-butylbenzoate. Non-limiting examples of styrene monomers include styrene and α-methylstyrene.

One of ordinary skill in the art will recognize that the at least one film-forming polymer may be formed from any monomers known to those skilled in the art which fall within the categories of acrylic and vinyl monomers (including monomers modified with a silicone chain). In one embodiment, the at least one film-forming polymer is not chosen from a vinyl-silicone graft or block copolymer comprising a silicone polymer segment and a vinyl polymer segment which is prepared by the free radical polymerization of a mercapto functional silicone chain transfer agent and vinyl monomers.

Non-limiting examples of acrylic film-forming polymers in aqueous dispersion which can be used according to the present invention include those sold by Zeneca under the tradenames Neocryl XK-90®, Neocryl A-1070®, Neocryl A-1090®, Neocryl BT-62®, Neocryl A-1079® and Neocryl A-523®, and those sold by Dow Chemical under the tradename Dow Latex 432®.

Suitable film-forming polymers formed via polycondensation which can be used as the at least one film-forming polymer may be anionic film-forming polymers, cationic film-forming polymers, nonionic film-forming polymers or amphoteric film-forming polymers. Non-limiting examples of suitable film-forming polymers formed via polycondensation include polyurethanes, polyurethane-acrylics, polyurethane-polyvinylpyrrolidones, polyester-polyurethanes, polyether-polyurethanes, polyureas and polyurea-polyurethanes.

Non-limiting examples of film-forming polyurethanes include aliphatic polyurethanes, cycloaliphatic polyurethanes, aromatic polyurethanes, polyurea-urethanes and polyurea copolymers. The film-forming polyurethanes may, for example, comprise at least one unit formed from polymerization of at least one monomer chosen from aliphatic monomers, cycloaliphatic monomers, aromatic polyester monomers, branched silicone monomers, non-branched silicone monomers, and monomers comprising at least one fluoro group.

Non-limiting examples of film-forming polyurethane polymers in aqueous dispersion include polyester-polyurethanes sold under the tradenames “Avalure UR-405®”, “Avalure UR-410®”, “Avalure UR-425®”, “Avalure UR-450®” and “Sancure 2060®” by Goodrich; polyether-polyurethanes sold under the tradename “Sancure 878®” by Goodrich; and polyether-polyurethanes sold under the tradename “Neorez R 970®” by Zeneca.

Non-limiting examples of film-forming polycondensates include polyesters, polyesteramides, fatty-chain polyesters, polyamides, epoxyester resins, resins formed from the condensation of formaldehyde with at least one arylsulphonamide, and arylsulphonamide epoxy resins. In one embodiment, the cosmetic compositions of the present invention do not further comprise both an arylene sulfide polymer and a ceramic microsphere filler.

Polyesters can, for example, be obtained by polycondensation of at least one dicarboxylic acid and at least one polyol such as at least one diol. The at least one dicarboxylic acid may, for example, be chosen from aliphatic dicarboxylic acids, alicyclic dicarboxylic acids, and aromatic dicarboxylic acids. Non-limiting examples of the at least one dicarboxylic acid include oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalenedicarboxylic acid and 2,6-naphthalene-dicarboxylic acid.

Polyols may, for example, be chosen from aliphatic polyols, alicyclic polyols, and aromatic polyols. Non-limiting examples of the at least one polyol include glycerol, pentaerythritol, sorbitol, and trimethylolpropane. Diols may, for example, be chosen from aliphatic diols, alicyclic diols, and aromatic diols. Non-limiting examples of the at least one diol include ethylene glycol, diethylene glycol, triethylene glycol, 1,3-propanediol, cyclohexanedimethanol, and 4-butanediol.

Polyesteramides may, for example, be obtained in a similar manner to the polyesters by polycondensation of at least one diacid and at least one diamine and/or at least one amino alcohol. Non-limiting examples of the at least one diamine include ethylenediamine, hexamethylenediamine, and meta-phenylenediamines, and para-phenylenediamine. A non-limiting example of the at least one amino alcohol is monoethanolamine.

Polyesters, for example, may further comprise at least one monomer which comprises at least one —SO₃M group, wherein M is chosen from H⁺, NH₄⁺ and metal cation such as, for example, Na⁺, Li⁺, K⁺, Mg²⁺, Ca²⁺, Cu²⁺, Fe²⁺, and Fe³⁺. A non-limiting example of the at least one monomer which comprises at least one —SO₃M group is a difunctional aromatic monomer comprising at least one —SO₃M group, wherein the at least one aromatic group may, for example, be chosen from benzene groups, naphthalene groups, anthracene groups, diphenyl groups, oxydiphenyl groups, sulphonyldiphenyl groups, and methylenediphenyl groups. Thus, non-limiting examples of difunctional aromatic monomers comprising at least one —SO₃M group include sulphoisophthalic acid, sulphoterephthalic acid, sulphophthalic acid, and 4-sulphonaphthalene-2,7-dicarboxylic acid.

Copolymers based on isophthalate/sulphoisophthalate, such as those formed from condensation of diethylene glycol, cyclohexanedimethanol, isophthalic acid, and sulphoisophthalic acid, may be used in the inventive compositions. Non-limiting examples of such polymers include those sold under the tradename Eastman AQ by Eastman Chemical Products.

As previously mentioned, the at least one film-forming polymer may be chosen from film-forming polymers of natural origin. Film-forming polymers of natural origin, as used herein, may optionally be modified. Non-limiting examples of film-forming polymers of natural origin include shellac resins, sandaraque gums, dammar resins, elemis gums, copal resins, and cellulose polymers such as nitrocelluloses, cellulose acetates, cellulose acetobutyrates, cellulose acetopropionates, and ethylcelluloses.

According to the present invention, the at least one film-forming polymer, if present, may be present in an amount generally ranging, for example, from 1% to 70% by weight, relative to the total weight of the composition, such as from 10% to 40%. One of ordinary skill in the art will recognize that the at least one film-forming polymer according to the present invention may be commercially available, and may come from suppliers in the form of a dilute solution. The amounts of the at least one film-forming polymer disclosed herein therefore reflect the weight percent of active material.

As used herein, a cosmetically acceptable medium is a medium which may be applied to at least one keratinous substance. In one embodiment, the cosmetically acceptable medium is chosen from at least one solvent, water and mixtures thereof. Non-limiting examples of the at least one solvent include organic solvents. Non-limiting examples of the cosmetically acceptable medium include aqueous-alcoholic mixtures such as mixtures comprising at least one C₁-C₅ monoalcohol.

Non-limiting examples of organic solvents include:

ketones which are liquid at room temperature (such as methyl ethyl ketone, methyl isobutyl ketone, diisobutyl ketone, isophorone, cyclohexanone, and acetone);

alcohols which are liquid at room temperature (such as ethanol, isopropanol, diacetone alcohol, 2-butoxyethanol, and cyclohexanol);

glycols which are liquid at room temperature (such as ethylene glycol, propylene glycol, pentylene glycol, and glycerol);

propylene glycol ethers which are liquid at room temperature (such as propylene glycol monomethyl ether, propylene glycol monomethyl ether acetate, and dipropylene glycol mono-n-butyl ether);

short-chain esters which comprise from 3 to 8 carbon atoms (such as ethyl acetate, methyl acetate, propyl acetate, n-butyl acetate, and isopentyl acetate) and are liquid at room temperature;

ethers which are liquid at room temperature (such as diethyl ether, dimethyl ether, and dichlorodiethyl ether);

alkanes which are liquid at room temperature (such as decane, heptane, dodecane, and cyclohexane);

cyclic aromatic compounds which are liquid at room temperature (such as toluene and xylene); and

aldehydes which are liquid at room temperature (such as benzaldehyde and acetaldehyde).

One of ordinary skill in the art will recognize that the cosmetically-acceptable medium, and therefore the at least one solvent, will be chosen according to the desired characteristics and application of the inventive composition. For example, certain solvents are known in the art to be suitable for certain applications, such as nail varnishes, mascara, etc.

According to the present invention, the cosmetically-acceptable medium may be present in an amount generally ranging from 30% to 99% by weight, relative to the total weight of the composition, such as from 60% to 90%.

The compositions of the present invention may further comprise at least one film-forming auxiliary agent. The at least one film-forming auxiliary agent may improve at least one film-forming property of the composition, such as at least one film-forming property chosen from at least one film-forming property of the base composition and at least one film-forming property of the surface composition. When at least one film-forming auxiliary agent is used with at least one film-forming polymer, the at least one film-forming auxiliary agent can be chosen from any compound known to those skilled in the art as being capable of modifying, such as enhancing, at least one property of the at least one film forming polymer.

In one embodiment, the at least one film-forming auxiliary agent is chosen from plasticizers. Plasticizers are used in the art for the purposes of softening and plasticizing the film-formers in order to provide better flexibility. Non-limiting examples of known plasticizers include tricresyl phosphate, benzyl benzoate, tributyl phosphate, butyl acetylricinoleate, glyceryl acetylricinoleate, dibutyl phthalate, butyl glycolate, dioctyl phthalate, butyl stearate, tributoxyethyl phosphate, triphenyl phosphate, triethyl citrate, tributyl acetylcitrate, 2-triethylhexyl acetylcitrate, dibutyl tartrate, dimethoxyethyl phthalate, diisobutyl phthalate, diamyl phthalate, camphor, glycerol triacetate, and glycerol tribenzoate.

In another embodiment, the at least one film-forming auxiliary agent is chosen from coalescers. For example, when the at least one film-forming polymer is in the form of particles dispersed in the at least one cosmetically acceptable medium, the at least one film-forming auxiliary agent may be chosen from coalescers.

The at least one film-forming auxiliary agent, if present, may be present in an amount generally ranging, for example, from 0.1% to 15% by weight relative to the total weight of the composition, such as from 0.5% to 10%.

According to the present invention, the composition may further comprise at least one coloring agent. Non-limiting examples of the at least one coloring agent include lipophilic dyes (liposoluble dyes), hydrophilic dyes, pulverulent dyestuffs including traditional pigments (including interferential and non-interferential pigments), nacres, glitters and flakes usually used in cosmetic or dermatological compositions, and mixtures thereof. The at least one coloring agent, if present, may be present in the composition in an amount generally ranging from 0.01% to 50% by weight, relative to the total weight of the composition, such as from 0.01% to 30%.

The composition according to the present invention may further comprise at least one additive known to those skilled in the art as being capable of being incorporated into cosmetic compositions. Non-limiting examples of the at least one additive include vitamins, minerals, thickeners, fillers, spreading agents, thixotropes, rheological agents, wetting agents, dispersants, anti-foaming agents, preserving agents, UV screening agents, active agents, surfactants, moisturizers, fragrances, neutralizing agents, stabilizers, proteins and antioxidants.

Needless to say, a person skilled in the art will take care to select the at least one suitable additive such that the advantageous properties of the composition in accordance with the invention are not, or are not substantially, adversely affected by the addition(s) envisaged.

The inventive compositions may, for example, be prepared by a sequential addition of all the ingredients or through several steps including the grinding of the nanoparticles in the presence of the pigments. One of ordinary skill in the art will recognize satisfactory equipment and procedures. For example, in the case of a nail varnish, the nanoparticles may also be mixed in a thixotrope such as a bentone gel.

In one embodiment, the present invention provides a cosmetic composition for at least one keratinous substance comprising coated nanoparticles in a cosmetically acceptable medium, wherein the coated nanoparticles are present in an amount effective to impart at least one long wear property to the cosmetic composition wherein the nanoparticles do not decrease the gloss of the composition.

In another embodiment, the present invention relates to a cosmetic process for making up at least one keratinous substance using the inventive composition. The composition according to the invention may, for example, be applied to the nails, facial or body skin, including the lips, the eyelashes, eyebrows, and/or the hair. The composition can also be applied to make-up accessories such as false nails, false eyelashes, wigs, and/or to pastilles or patches adhering to the skin and/or the lips such as beauty spots.

In another embodiment, the present invention is drawn to a nail varnish product, a face powder product, an eyeshadow product, a foundation product, a mascara product, an eyeliner product, or a make-up product for the lips and/or body comprising nanoparticles in an amount effective to impart at least one long wear property to the product, wherein the nanoparticles do not decrease the gloss of the composition. In one embodiment, the inventive composition is a nail varnish. In another embodiment, the nanoparticles are coated nanoparticles.

In yet another embodiment, the present invention is drawn to a cosmetic composition for at least one keratinous substance comprising, in a cosmetically acceptable medium, (i) at least one film-forming polymer and (ii) nanoparticles, wherein the nanoparticles are present in an amount effective to impart at least one long wear property to the cosmetic composition, wherein the nanoparticles do not decrease the gloss of the composition. In another embodiment, the nanoparticles are coated nanoparticles.

In yet another embodiment, the present invention is drawn to a cosmetic composition for at least one keratinous substance comprising nanoparticles in a cosmetically acceptable medium, wherein the nanoparticles are present in an amount effective to impart at least one long wear property to the cosmetic composition, wherein the nanoparticles do not decrease the gloss of the composition, and further wherein the nanoparticles form a concentrated layer of nanoparticles at the surface of the composition on the at least one keratinous substance.

In yet another embodiment, the present invention is drawn to a process for making a cosmetic composition for at least one keratinous substance comprising including in the cosmetic composition nanoparticles in an amount effective to impart at least one long wear property to the cosmetic composition, wherein the nanoparticles do not decrease the gloss of the composition.

In yet another embodiment, the present invention provides a coated substrate comprising a substrate and a cosmetic composition comprising nanoparticles in an amount effective to impart at least one long wear property to the cosmetic composition, wherein the nanoparticles do not decrease the gloss of the composition. In one embodiment, the substrate is at least one keratinous substance. In another embodiment, the substrate is a make-up accessory such as those discussed above. In another embodiment, the nanoparticles form a concentrated layer of nanoparticles in the cosmetic composition on the substrate.

Another embodiment of the present invention relates to a coated false nail, false eyelash, pastille, patch adhering to the skin or the lips, or a wig resulting from the application thereto of a cosmetic composition comprising nanoparticles in an amount effective to impart at least one long wear property to the cosmetic composition to the false nail, false eyelash, pastille, patch adhering to the skin or the lips, or wig.

In another embodiment, the present invention is directed to a method for improving gloss of a film of a cosmetic composition comprising including nanoparticles in the cosmetic composition in an amount effective to impart at least one long wear property to the cosmetic composition.

In yet another embodiment, the present invention provides a process of making a cosmetic composition resistant to wear comprising including nanoparticles in the cosmetic composition in an amount effective to impart at least one long wear property to the cosmetic composition.

One embodiment of the present invention relates to a method for improving hardness of a film of a cosmetic composition comprising including nanoparticles in the cosmetic composition in an amount effective to impart at least one long wear property to the cosmetic composition.

Another embodiment of the present invention is drawn to a process for a making a cosmetic composition resistant to wear comprising the steps of blending nanoparticles with at least one Theological agent.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and in the attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. The following examples are intended to illustrate the invention without limiting the scope as a result. The percentages are given on a weight basis.

### EXAMPLES

### Example 1

Five nail varnishes, Sample A (Control), B (Comparative) C (Inventive), D (Inventive) and E (Inventive) were prepared as follows:

| Ingredient | A | B | C | D | E |
|---|---|---|---|---|---|
| Nitrocellulose | 9.11 | 8.5 | 9.29 | 6.42 | 6.56 |
| Epoxy | 1.44 | 1.44 | 1.44 | 1.44 | 1.44 |
| Polyester | 1.5 | 1.5 | 1.5 | 1.5 | 1.5 |
| Acrylic | 1.2 | 1.2 | 1.2 | 1.2 | 1.2 |
| Plasticizer | 5.49 | 5.46 | 5.46 | 5.46 | 5.46 |
| UV Absorber | 0.4 | 0.4 | 0.4 | 0.4 | 0.4 |
| Bentone | 3.0 | 3.0 | 3.0 | 3.0 | 3.0 |
| Pigments | 2.5 | 2.5 | 2.5 | 2.5 | 2.5 |
| Coated | 0 | 0 | 3.35 | 6.71 | 10.03 |
| Nanoparticles | | | | | |
| Microspheres¹ | 0 | 1.0 | 0 | 0 | 0 |
| Solvents | 84.36 | 75.00 | 71.86 | 71.47 | 67.81 |
| Hardness | 84 | 87 | 186 | 175 | 150 |
| Gloss | 69 | 43 | 70 | 80 | 70 |

¹W210 Zeeospheres from 3M and Zeelan Industries, St. Paul, MN have a particle size distribution corresponding to 325 mesh (less than 1 micron to about 40 microns).

The coated nanoparticles were aluminum oxide nanoparticles coated with 2% of triphenyl phosphate. The coated nanoparticles had an average size of 37 nm and a size distribution of 26 nm to 50 nm.

Sample A was the control composition and contained no microspheres and no nanoparticles. Sample B was a comparative composition and contained microspheres, but no nanoparticles. Samples C, D, and E each contained a different amount of nanoparticles. The hardness of each sample was measured using the Persoz Pendulum Hardness Test (ASTM D4366-95), while the gloss for each sample was measured using the Standard Test Method for Specular Gloss, ASTM method D523-89.

The results demonstrate that the addition of microspheres lowers the gloss of a nail varnish and imparts no appreciable change in hardness (sample B). The addition of nanoparticles to a nail varnish, however, provides a significant increase in hardness while not affecting the gloss and in some cases increasing the gloss.

What is claimed is:

1. A cosmetic composition for at least one keratinous substance comprising nanoparticles in a cosmetically acceptable medium, wherein said nanoparticles are present in an amount effective to impart at least one long wear property to said cosmetic composition, and further wherein said nanoparticles do not decrease the gloss of said composition.

2. The composition according to claim 1, wherein said at least one long wear property are chosen from resistance to impacts, resistance to rubbing, resistance to scuffing, resistance to abrasion, and resistance to chipping.

3. The composition according to claim 1, wherein said nanoparticles have an average particle size of less than 800 nanometers

4. The composition according to claim 3, wherein said nanoparticles have an average particle size ranging from 10 nm to 500 nm.

5. The composition according to claim 4, wherein said nanoparticles have an average particle size ranging from 30 nm to 100 nm.

6. The composition according to claim 1, wherein said nanoparticles are chosen from spherical nanoparticles, spheroidal nanoparticles and tubular nanoparticles.

7. The composition according to claim 1, wherein said nanoparticles are chosen from metallic powders, inorganic powders, nanoparticles formed from at least one composite polymer, and nanopolymers formed from at least one clay.

8. The composition according to claim 7, wherein said metallic powders are formed from at least one metal.

9. The composition according to claim 8, wherein said at least one metal is chosen from silver, gold, platinum, palladium, and nickel.

10. The composition according to claim 7, wherein said inorganic powders are chosen from inorganic powders formed from at least one inorganic oxide.

11. The composition according to claim 10, wherein said at least one inorganic oxide is chosen from oxides of magnesium, oxides of calcium, oxides of strontium, oxides of yttrium, oxides of chromium, oxides of molybdenum, oxides of vanadium, oxides of titanium, oxides of holmium, oxides of cerium, oxides of neodymium, oxides of iron, oxides of copper, oxides of aluminum, oxides of indium, oxides of silicon, oxides of tin, oxides of antimony, and mixtures of any of the foregoing.

12. The composition according to claim 1, wherein said nanoparticles are formed from at least one metal chosen from aluminum, silver, nickel, chromium and molybdenum.

13. The composition according to claim 1, wherein said nanoparticles are coated with at least one material chosen from materials that will change the polarity of said nanoparticles and materials that will change the hydrophobicity of said nanoparticles.

14. The composition according to claim 1, wherein said nanoparticles are coated with at least one material that will lower the surface free energy of said nanoparticles.

15. The composition according to claim 14, wherein said at least one material that will lower the surface free energy of said nanoparticles is chosen from phosphates, fluorinated compounds, silicone compounds, and hydrocarbons.

16. The composition according to claim 15, wherein said phosphates are chosen from triphenyl phosphate.

17. The composition according to claim 15, wherein said fluorinated compounds are chosen from perfluoroethers and derivatives thereof.

18. The composition according to claim 15, wherein said silicone compounds are chosen from polysilicone-8.

19. The composition according to claim 1, wherein said nanoparticles are present in an amount ranging from 0.01% to 50% by weight, relative to the total weight of the composition.

20. The composition according to claim 19, wherein said nanoparticles are present in an amount ranging from 0.1% to 40% by weight, relative to the total weight of the composition.

21. The composition according to claim 20, wherein said nanoparticles are present in an amount ranging from 1% to 30% by weight, relative to the total weight of the composition.

22. The composition according to claim 21, wherein said nanoparticles are present in an amount ranging from 1.5% to 20% by weight, relative to the total weight of the composition.

23. The composition according to claim 22, wherein said nanoparticles are present in an amount ranging from 2% to 10% by weight, relative to the total weight of the composition.

24. The composition according to claim 23, wherein said nanoparticles are present in an amount ranging from 3% to 8% by weight, relative to the total weight of the composition.

25. The composition according to claim 1, further comprising at least one film-forming polymer.

26. The composition according to claim 25, wherein said at least one film-forming polymer is dissolved in said cosmetically acceptable medium.

27. The composition according to claim 25, wherein said at least one film-forming polymer is dispersed in said cosmetically acceptable medium.

28. The composition according to claim 25, wherein the at least one film-forming polymer is chosen from synthetic film-forming polymers and film-forming polymers of natural origin.

29. The composition according to claim 28, wherein said synthetic film-forming polymers are chosen from film-forming polymers formed via radical-mediated polymerization and film-forming polymers formed from polycondensation.

30. The composition according to claim 25, wherein the at least one film-forming polymer is chosen from vinyl polymers, acrylic polymers, polyurethanes, polyureas, polyesters, polyesteramides, polyamides, epoxyester resins, resins formed from condensation of formaldehyde with at least one arylsulphonamide, arylsulphonamide epoxy resins, shellac resins, sandaraque gums, dammar resins, elemis gums, copal resins and cellulose polymers.

31. The composition according to claim 30, wherein said polyurethanes are chosen from polyurethane-acrylics, polyurethane-polyvinyl pyrrolidones, polyester-polyurethanes, polyether-polyurethanes, and polyurea-polyurethanes.

32. The composition according to claim 30, wherein said polyesters are chosen from fatty-chain polyesters.

33. The composition according to claim 30, wherein said vinyl polymers are chosen from acrylic polymers.

34. The composition according to claim 30, wherein said cellulose polymers are chosen from nitrocelluloses, cellulose acetates, cellulose acetobutyrates, cellulose acetopropionates and ethylcelluloses.

35. The composition according to claim 25, wherein said at least one film-forming polymer is present in an amount ranging from 1% to 70% by weight, relative to the total weight of the composition.

36. The composition according to claim 35, wherein said at least one film-forming polymer is present in an amount ranging from 10% to 40% by weight, relative to the total weight of the composition.

37. The composition according to claim 1, wherein said cosmetically acceptable medium is chosen from at least one solvent, water and mixtures thereof.

38. The composition according to claim 14, wherein said at least one solvent is chosen from organic solvents.

39. The composition according to claim 1, wherein the cosmetically acceptable medium is chosen from aqueous-alcoholic mixtures.

40. The composition according to claim 39, wherein said aqueous-alcoholic mixture comprises at least one C₁-C₅ monoalcohol.

41. The composition according to claim 38, wherein said organic solvents are chosen from ketones which are liquid at room temperature; alcohols which are liquid at room temperature; glycols which are liquid at room temperature; propylene glycol ethers which are liquid at room temperature; short-chain esters which comprise from 3 to 8 carbon atoms and are liquid at room temperature; ethers which are liquid at room temperature; alkanes which are liquid at room temperature; cyclic aromatic compounds which are liquid at room temperature; and aldehydes which are liquid at room temperature.

42. The composition according to claim 1, wherein said cosmetically acceptable medium is present in an amount ranging from 30% to 99% by weight, relative to the total weight of the composition.

43. The composition according to claim 42, wherein said cosmetically acceptable medium is present in an amount ranging from 60% to 90% by weight, relative to the total weight of the composition.

44. The composition according to claim 1, further comprising at least one film-forming auxiliary agent.

45. The composition according to claim 44, wherein said at least one film-forming auxiliary agent is chosen from plasticizers and coalescers.

46. The composition according to claim 45, wherein said plasticizers are chosen from tricresyl phosphate, benzyl benzoate, tributyl phosphate, butyl acetylricinoleate, glyceryl acetylricinoleate, dibutyl phthalate, butyl glycolate, dioctyl phthalate, butyl stearate, tributoxyethyl phosphate, triphenyl phosphate, triethyl citrate, tributyl acetyl citrate, 2-triethylhexyl acetylcitrate, dibutyl tartrate, dimethoxyethyl phthalate, diisobutyl phthalate, diamyl phthalate, camphor, glycerol triacetate, and glycerol tribenzoate.

47. The composition according to claim 44, wherein said at least one film-forming auxiliary agent is present in an amount ranging from 0.1% to 15% by weight, relative to the total weight of the composition.

48. The composition according to claim 47, wherein said at least one film-forming auxiliary agent is present in an amount ranging from 0.5% to 10% by weight, relative to the total weight of the composition.

49. The composition according to claim 1, further comprising at least one coloring agent.

50. The composition according to claim 49, wherein the at least one coloring agent is chosen from lipophilic dyes, hydrophilic dyes, and pulverulent dyestuffs.

51. The composition according to claim 49, wherein the at least one coloring agent is present in an amount ranging from 0.01% to 50% by weight, relative to the total weight of the composition.

52. The composition according to claim 51, wherein the at least one coloring agent is present in an amount ranging from 0.01% to 30% by weight, relative to the total weight of the composition.

53. The composition according to claim 1, further comprising at least one additive chosen from vitamins, minerals, thickeners, fillers, spreading agents, thixotropes, rheological agents, wetting agents, dispersants, anti-foaming agents, preserving agents, UV-screening agents, active agents, surfactants, moisturizers, fragrances, neutralizing agents, stabilizers, and antioxidants.

54. A cosmetic composition for at least one keratinous substance comprising coated nanoparticles in a cosmetically acceptable medium, wherein the coated nanoparticles are present in an amount effective to impart at least one long wear property to said cosmetic composition, and further wherein said nanoparticles do not decrease the gloss of said composition.

55. The composition according to claim 1, wherein said composition is a nail varnish product, a face powder product, an eyeshadow product, a foundation product, a mascara product, an eyeliner product, a make-up product for the lips, or a make-up product for the body.

56. The composition according to claim 55, wherein said nanoparticles are coated nanoparticles.

57. A cosmetic composition for at least one keratinous substance comprising, in a cosmetically acceptable medium,
(i) nanoparticles and
(ii) at least one film-forming polymer,
wherein said nanoparticles are present in an amount effective to impart at least one long wear property to said cosmetic composition, and further wherein said nanoparticles do not decrease the gloss of said composition.

58. The cosmetic composition according to claim 57, wherein said nanoparticles are coated nanoparticles.

59. A cosmetic composition for at least one keratinous substance comprising nanoparticles in a cosmetically acceptable medium, wherein said nanoparticles are present in an amount effective to impart at least one long wear property to said cosmetic composition, wherein said nanoparticles do not decrease the gloss of said composition, and further wherein said nanoparticles form a concentrated layer of nanoparticles in said cosmetic composition on said at least one keratinous substance.

60. The cosmetic composition according to claim 59, wherein said nanoparticles are coated nanoparticles.

61. A process for forming a concentrated layer of nanoparticles at a surface of a cosmetic composition on at least one keratinous substance comprising applying to at least one keratinous substance a cosmetic composition comprising nanoparticles in a cosmetically acceptable medium.

62. A process for making up, caring or treating at least one keratinous substance comprising applying to said at least one keratinous substance a cosmetic composition comprising nanoparticles, wherein said nanoparticles are present in an amount effective to impart at least one long wear property to said cosmetic composition, and further wherein said nanoparticles do not decrease the gloss of said composition.

63. A coated substrate comprising a substrate and a cosmetic composition comprising nanoparticles in an amount effective to impart at least one long wear property to said cosmetic composition, wherein said nanoparticles do not decrease the gloss of said composition.

64. The coated substrate according to claim 63, wherein said nanoparticles form a concentrated layer of nanoparticles in said cosmetic composition on said substrate.

65. A coated false nail, false eyelash, pastille, patch adhering to the skin or the lips, or a wig resulting from the application thereto of a cosmetic composition comprising nanoparticles in an amount effective to impart at least one long wear property to said cosmetic composition, wherein said nanoparticles do not decrease the gloss of said false nail, false eyelash, pastille, patch adhering to the skin or the lips, or wig.
